(19) United States
(12) Patent Application Publication
(10) Pub. No.: US 2016/0235994 A1
(43) Pub. Date: Aug. 18, 2016

(54) DEVICE FOR FACILITATING BLOOD CIRCULATION BY HEAT AND ELECTROSHOCK AND METHOD USING THEREOF

(71) Applicant: HITOMI DOI, OSAKA (JP)

(72) Inventor: HITOMI DOI, OSAKA (JP)

(21) Appl. No.: 14/923,808

(22) Filed: Oct. 27, 2015

(30) Foreign Application Priority Data
Feb. 16, 2015 (TW) 104202572

(51) Int. Cl. A61N 1/38; A61F 7/08

(52) U.S. Cl. CPC A61N 1/38; A61F 7/08

(57) ABSTRACT

A device for facilitating blood circulation by heat and electroshock and a method using thereof are provided. The device includes a host, one or more electroshock assembly, and two or more heat pads. The host has a control circuit to generate a direct current power. The electroshock assembly is electrically connected to the host and includes electroshock members as positive and negative electrodes. The electroshock members are attached to two opposite sides of a portion of a human body, so that the output current generated by the host is transmitted from the positive electrode, through the portion of the human body, to the negative electrode. The heat pads are covered on the outer surfaces of the electroshock members when the electroshock members are attached to the human body, so that the heat can be transmitted to the interior portion of the human body.

### BACKGROUND OF THE INVENTION

1. Field of the Invention

The present invention relates to a device for facilitating blood circulation by heat and electroshock and method using thereof, and more particularly to a device capable of transmitting heat to interior portions of a user's body and method using thereof.

2. Description of the Prior Art

As shown in FIG. 5, an existing massage device is illustrated. The device has a host 91 and two electrode plates 92 respectively connected to the host 91 by wires 93. When in use, firstly the electrode plates 92 are attached to a portion of a user. Then, the massage mode can be set with the host 91. When the host 91 is turned on, the host 91 outputs low-frequency currents to the electrode plates 92, and the electrode plates 92 transmit the current to stimulate the skin and superficial muscles of the user, so that the user would feel his/her skin get slight electric shock and unconsciously retract his/her muscle. Accordingly, the device performs a massage function to allow the user's body to relax.

However, since the low-frequency currents generated by the host 91 and transmitted by the electrode plates 92 can be only delivered to the superficial part of the user's body, and cannot be further delivered to the interior portions of the user's body, such as deep muscle groups, nerves, arteries, and veins, the massage function provided by the device would be limited. In addition, when too much electric shocks are applied to the skin and the superficial muscles of the user, sometimes the user may feel skin uncomfortable or muscle over-retracted, or has other adverse effects.

The present invention is, therefore, arisen to obviate or at least mitigate the above mentioned disadvantages.

### SUMMARY OF THE INVENTION

One object of the present invention is to solve the aforementioned problems and provide a device for facilitating blood circulation by heat and electroshock and method using thereof. The device and the method allow the heat generated by the heat pads to be transmitted to the interior portions of a human body along with the output current generated by the host of the device. Therefore, a blood circulation function can be approached.

To achieve the above and other objects, a device for facilitating blood circulation by heat and electroshock comprises a host, at least one electroshock assembly, and at least two heat pads. The host has a control circuit for generating direct current power. The direct current power comprises an output voltage and an output current. The output voltage is 7 to 9 volts and the frequency of the output voltage is 50 to 60 Hz. The output current is 100 to 300 mA. The control circuit applies filtering to the direct current power so as to allow the direct current power to have a bandwidth-fixed and smoothed waveform. The electroshock assembly is electrically connected to the host via wires. The electroshock assembly comprises a first electroshock member and a second electroshock member. The first electroshock member and the second electroshock member are of plate shape. One of the two electroshock members is a positive electrode, and the other is a negative electrode. The first electroshock member and the second electroshock member are respectively adapted to be attached on opposite sides of a portion of a user's body. The output generated by the host is transmitted from the positive electrode, through the user's body, to the negative electrode. The heat pads are adapted to be covered on outer surfaces of the electroshock members when the electroshock members are attached on the user's body.

Wherein, each of the electroshock members has a conductive layer and a bag. The conductive layer is enclosed by the bag, and the bag is adapted to be bedewed prior to attaching to the user's body.

Wherein, the heat pads are made of materials selected from neoprene, polyethylene, or polyester to allow heat in the heat pads to be transmitted out of the heat pads slowly.

Wherein, the host has an operating interface. The operating interface comprises a power switch portion, a time control portion, a current control portion, and a polarity switch portion. The power switch portion is for turning on or off the host. The time control portion is for setting an operation time to the host. The current control portion is for setting the value of the output current. The polarity switch portion is for changing the polarities of the electroshock members so as to change the direction of the output current.

Wherein, the time control portion comprises a knob and a monitor. The knob is adapted to set the operation time, and the monitor is adapted to display the operation time set by the knob.

Wherein, the current control portion comprises a knob and a monitor. The knob is adapted to set the value of the output current, and the monitor is adapted to display the value of the output current set by the knob.

Furthermore, a method for using the device for facilitating blood circulation by heat and electroshock comprising choosing a particular portion from the head portion, the neck portion, one of the four limbs, and the trunk of a user's body for applying the device, attaching the first electroshock member to one of two opposite sides of the particular portion, attaching the second electroshock member to the other side of the particular portion, respectively covering the heat pads on the outer surfaces of the electroshock members, turning on the host to generate the output voltage and the output current, and allowing the output current to be transmitted from the positive electrode of the electroshock members to the negative electrode of the electroshock members through the particular portion, so that the heat generated by the heat pads are transmitted along with the output current so as to pass through the particular portion and is further transmitted to interior portions of the user's body.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 illustrates a schematic perspective view of a device for facilitating blood circulation by heat and electroshock according to the present invention;

FIG. 2 illustrates a plan view of an operating interface of the device according to the present invention;

FIG. 3 illustrates a schematic operational view showing the device is applied to the abdomen of a user according to a first embodiment of the present invention;

FIG. 4 illustrates a schematic operational view showing the device is applied to the arm of a user according to a second embodiment of the present invention; and

FIG. 5 illustrates a schematic operational view of an existing massage device.

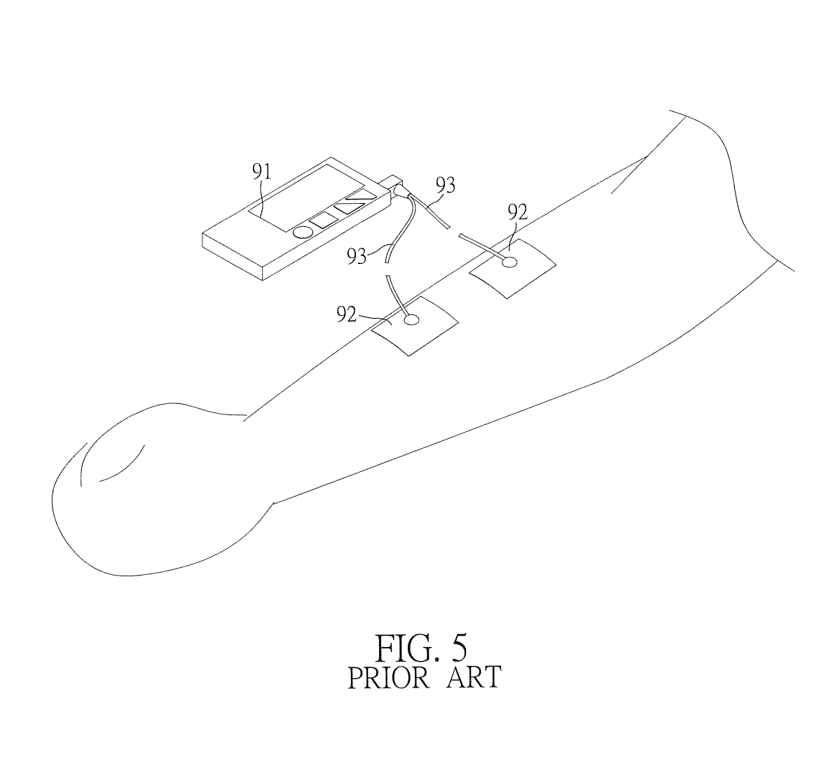

### DETAILED DESCRIPTION OF THE PREFERRED EMBODIMENTS

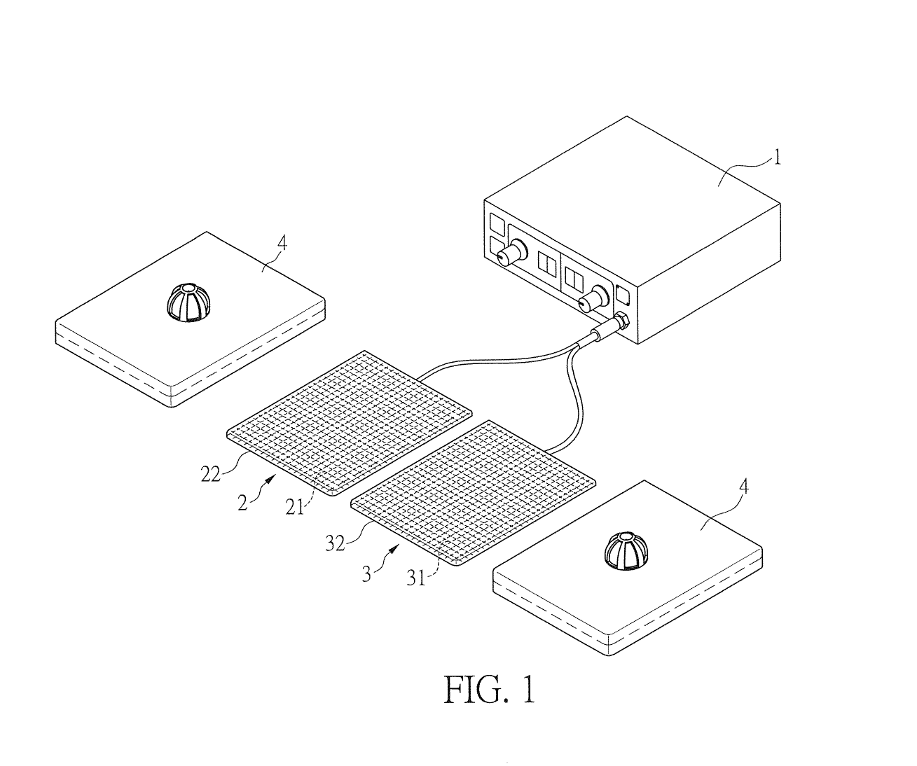

Please refer to FIGS. 1 to 4, illustrating embodiments of a device for facilitating blood circulation by heat and electroshock. The figures are provided for illustrative purposes and not limitations to the present invention.

In his embodiment, a device for facilitating blood circulation by heat and electroshock and method using thereof are provided. As shown in FIG. 1, the device comprises a host a, a first electroshock member 2, a second electroshock member 3, and two heat pads 4.

The host 1 shown in FIG. 1 has a control circuit for generating direct current power. The direct current power comprises an output voltage and an output current. The output voltage of the direct current power is 7 to 9 volts, the frequency of the output voltage is 50 to 60 Hz, and the output current of the direct current power is 100 to 300 mA. The control circuit applies filtering to the direct current power so as to allow the direct current power to have a bandwidth-fixed and smoothed waveform.

As shown in FIG. 1, in this embodiment, the first electroshock member 2 and the second electroshock member 3 are formed as an electroshock assembly. Both the first electroshock member 2 and the second electroshock member 3 are of plate shape. The first electroshock member 2 and the second electroshock member 3 are electrically connected to the host 1 via wires. One of the two electroshock members is a positive electrode, and the other electroshock member is a negative electrode. The heat pads 4 are respectively covered on the outer surfaces of the electroshock members 2, 3 when the electroshock members 2, 3 are attached to a human body. In this embodiment, each of the electroshock members 2, 3 has a conductive layer 21, 31 and a bag 22, 32 adapted to enclose the conductive layer 21, 31. The bag 22, 32 is adapted to be bedewed prior to attaching to the user's body. In addition, the heat pads 4 are made of materials selected from neoprene, polyethylene, or polyester to allow heat in the heat pads 4 to be transmitted out of the heat pads 4 slowly.

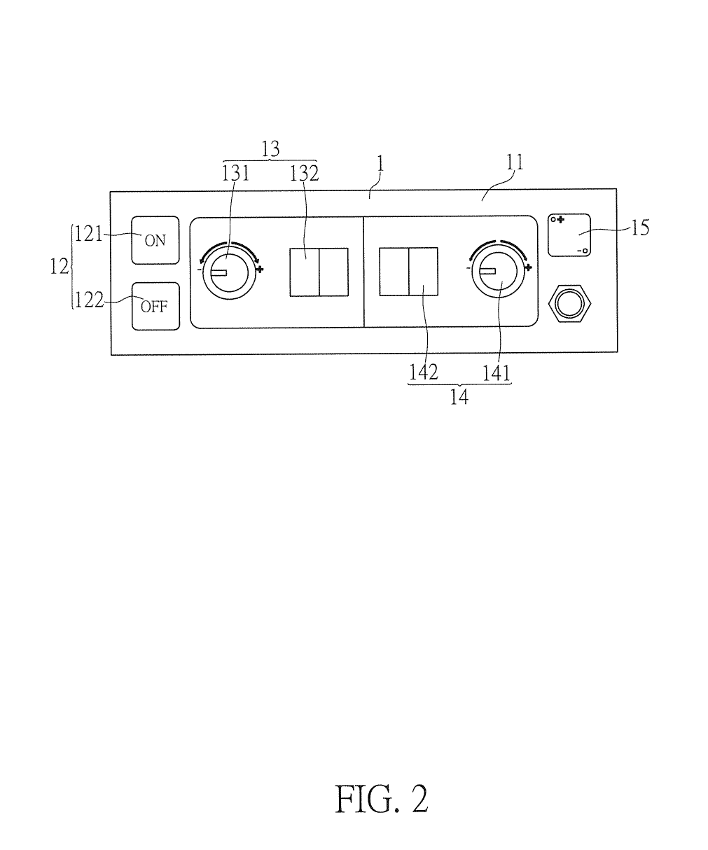

As shown in FIGS. 1 to 2, in this embodiment, the host 1 has an operating interface 11. The operating interface 11 comprises a power switch portion 12, a time control portion 13, a current control portion 14, and a polarity switch portion 15. In this embodiment, the power switch portion 12 comprises a turn-on button 121 and a turn-off button 122. With the operation of the turn-on button 121 and the turn-off button 122, the host 1 can be turned on or off. In this embodiment, the time control portion 13 comprises a knob 131 and a monitor 132. The knob 131 is adapted to set the operation time, and the monitor 132 is adapted to display the operation time set by the knob 131. In this embodiment, the current control portion 14 comprises a knob 141 and a monitor 142. The knob 141 is adapted to set the value of the output current, and the monitor 142 is adapted to display the value of the output current set by the knob 141. The polarity switch portion 15 is for changing the direction of the output current between the first electroshock member 2 and the second electroshock member 3. That is, the polarity of the first electroshock member 2 and the polarity of the second electroshock member 3 can be exchanged by the polarity switch portion 15, so that the direction of the output current is changed to an opposite direction.

In this embodiment, the control circuit of the host 1 is a digital control circuit, and the operation time set by the time control portion 13 and the value of the output current set by the current control portion 14 are zeroed when the control circuit is restarted by the power switch portion 12. In other words, after the host 1 is turned off, the control circuit does not memorize the operation time, the value of the output current, or other parameters; alternately, the user resets the operation time and the value of the output current after the host 1 is restarted. Accordingly, by the reset function, once the value of the output current applied to a former user is too high, a latter user would not have to use the parameters set by the former user and would not feel uncomfortable.

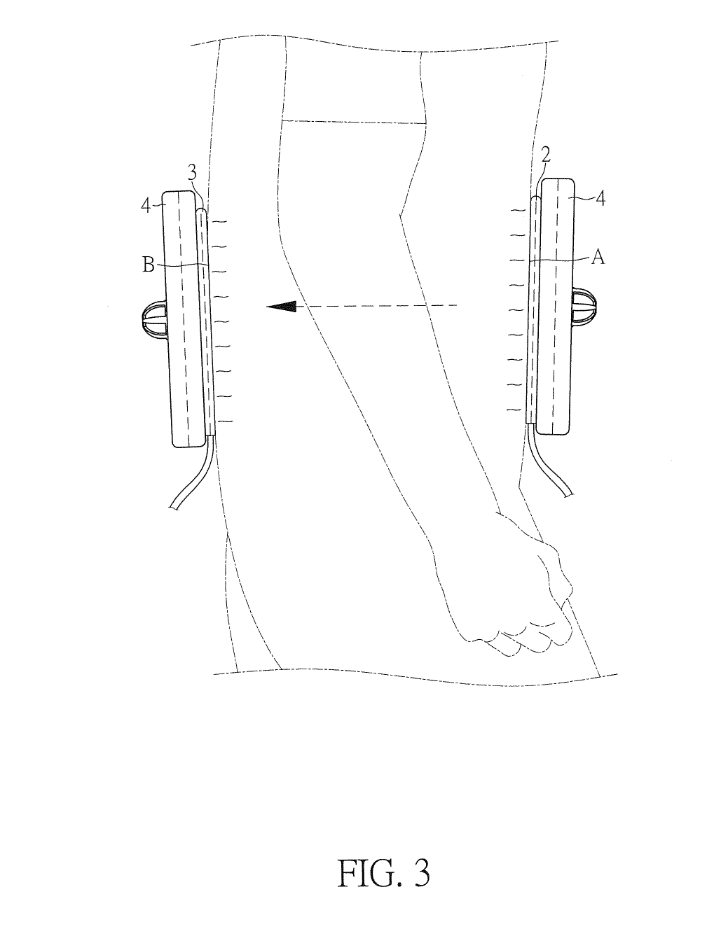

In this embodiment, a method for using the device is described. Firstly, a particular portion is chosen from the head portion, the neck portion, the four limbs, and the trunk of a user's body for applying the device. In this embodiment, the particular portion is the trunk of the user's body. And, as shown in FIG. 3, the first electroshock member 2 is provided as the positive electrode and attached to the abdomen portion A of the user's body, and the second electroshock member 3 is provided as the negative electrode and attached to a portion of the user's body opposite to the first electroshock member 2, i.e., attached to the back portion B of the user's body which is at the same height with respect to the abdomen portion A. Next, the heat pads 4 are respectively covered on the outer surfaces of the first electroshock members 2 and the second electroshock members 3. Then, the host 1 is turned on by the power switch portion 12 so as to allow the control circuit to generate the output voltage and the output current, thus allowing the output current to be transmitted from the first electroshock member 2, through the portion between the abdomen portion A and the back portion B, to the second electroshock member 3. Hence, the heat generated by the heat pads 4 are transmitted along with the output current so as to pass through the portion between the abdomen portion A and the back portion B and is further transmitted to the deep muscles, the nerves, the blood vessels, the organs, or other interior portions of the user's body.

If the user wants to change the current direction, i.e., to allow the output current transmitted from the second electroshock member 3 to the first electroshock member 2, the user can operate the polarity switch portion 15 to change the polarity of the first electroshock member 2 to be a negative electrode and the polarity of the second electroshock member 3 to be a positive electrode. Accordingly, the direction of the output current between the first electroshock member 2 and the second electroshock member 3 is from the back portion B to the abdomen portion A.

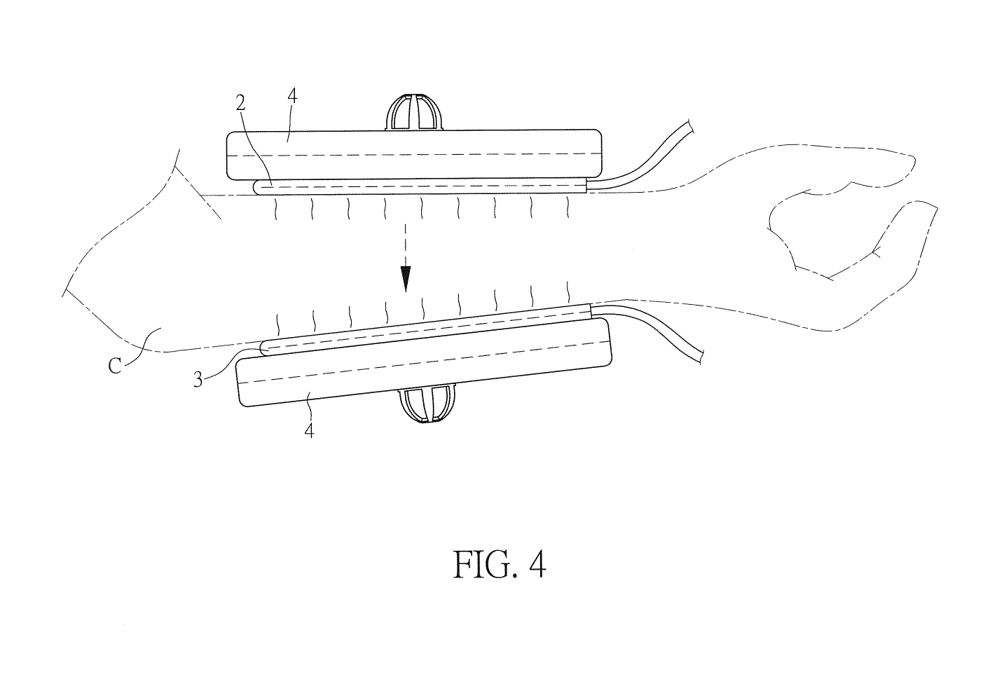

Further, as shown in FIG. 4, which illustrates a schematic operational view showing the device is applied to the arm C of a user according to a second embodiment of the present invention. In detail, the first electroshock member 2 is attached to a top portion of the arm C, and the second electroshock member 3 is attached to a bottom portion of the arm C. Similarly, the heat pads 4 are respectively covered on the outer surfaces of the first electroshock member 2 and the second electroshock member 3. Then, the host 1 is turned on by the power switch portion 12 so as to generate the output voltage and the output current, thus allowing the output current to be transmitted from the positive electrode, i.e., the first electroshock member 2, through the arm C, to the negative electrode, i.e., the second electroshock member 3. Hence, the heat generated by the heat pads 4 are transmitted along with the output current so as to pass through the arm C and is further transmitted to the interior portions of the arm C of the user.

Based on the above, the present invention has following advantages.

Firstly, according to the device and the method of the present invention, the electroshock assembly can be attached to a user's body along with the heat pads 4, so that the heat and the current can be transmitted to the interior portions of the body. As compared to the existing massage device which can stimulate the skin and superficial muscles of the user's body, the device according to the present invention can further stimulate the deep muscles, the nerves, the blood vessels, the organs, or other interior portions of the user's body. Thus, the device can facilitate the blood circulation.

Secondly, since the first electroshock member 2 and the second electroshock member 3 are respectively attached to the two opposite sides of the particular portion, the output current is transmitted between the first electroshock member 2 and the second electroshock member 3. Therefore, the user would not feel his/her skin get slight electric shock and unconsciously retract his/her muscle; instead, the user may feel that the heat is transmitted to interior portions of his/her body. Accordingly, the user would not feel skin hurt, muscle over-retracted or other adverse effects.

According to the descriptions from testees who have experienced the device, when the device is used with the method mentioned in the present invention, the testee feels relax and the fatigues be dissipated because of the output current between the first electroshock member 2 and the second electroshock member 3. After the application of the device, the testee would feel the portion of his/her body applied by the device get become warmer which is comfortable and consistent, and this is because of the blood capillary expansion, and congestion conditions occur which facilitate the blood circulation. Along with the aforementioned effects, the testee also feels energetic, and can have good sleep and good appetite.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A device for facilitating blood circulation by heat and electroshock, comprising:
a host having a control circuit for generating direct current power, wherein the direct current power comprises an output voltage and an output current, the output voltage is 7 to 9 volts and the frequency of the output voltage is 50 to 60 Hz, the output current is 100 to 300 mA, and the control circuit applies filtering to the direct current power so as to allow the direct current power to have a bandwidth-fixed and smoothed waveform;
at least one electroshock assembly electrically connected to the control circuit of the host by wires, wherein the electroshock assembly comprises a first electroshock member and a second electroshock member, the first electroshock member and the second electroshock member are of plate shape, one of the electroshock members is a positive electrode, and the other is a negative electrode, the first electroshock member and the second electroshock member are respectively adapted to be attached on opposite sides of a portion of a user's body, and the output current generated by the host is transmitted from the positive electrode, through the user's body, to the negative electrode; and
at least two heat pads adapted to be covered on outer surfaces of the electroshock members.

2. The device for facilitating blood circulation by heat and electroshock according to claim 1, wherein each of the electroshock members has a conductive layer and a bag, the conductive layer is enclosed by the bag, and the bag is adapted to be bedewed prior to attaching to the user's body.

3. The device for facilitating blood circulation by heat and electroshock according to claim 1, wherein the heat pads are made of materials selected from neoprene, polyethylene, or polyester to allow heat in the heat pads to be transmitted out of the heat pads slowly.

4. The device for facilitating blood circulation by heat and electroshock according to claim 1, wherein the host has an operating interface, the operating interface comprises a power switch portion, a time control portion, a current control portion, and a polarity switch portion, the power switch portion is for turning on or off the host, the time control portion is for setting an operation time to the host, the current control portion is for setting the value of the output current, and the polarity switch portion is for changing the polarities of the electroshock members so as to change the direction of the output current.

5. The device for facilitating blood circulation by heat and electroshock according to claim 4, wherein the time control portion comprises a knob and a monitor, the knob is adapted to set the operation time, and the monitor is adapted to display the operation time set by the knob.

6. The device for facilitating blood circulation by heat and electroshock according to claim 4, wherein the current control portion comprises a knob and a monitor, the knob is adapted to set the value of the output current, and the monitor is adapted to display the value of the output current set by the knob.

7. The device for facilitating blood circulation by heat and electroshock according to claim 4, wherein the control circuit of the host is a digital control circuit, the operation time set by the time control portion of the operation interface and the value of the output current set by the current control portion of the operation interface are zeroed when the control circuit is restarted by the power switch portion, so that a user can reset the operation time and the value of the output current.

8. A method for using the device for facilitating blood circulation by heat and electroshock according to claim 1, comprising: choosing a particular portion from the head portion, the neck portion, one of the four limbs, and the trunk of a user's body for applying the device, attaching the first electroshock member to one of two opposite sides of the particular portion, attaching the second electroshock member to the other side of the particular portion, respectively covering the heat pads on the outer surfaces of the electroshock members, turning on the host to generate the output voltage and the output current, and allowing the output current to be transmitted from the positive electrode of the electroshock members to the negative electrode of the electroshock members through the particular portion, so that the heat generated by the heat pads are transmitted along with the output current so as to pass through the particular portion and is further transmitted to interior portions of the user's body.
